(19) United States
(12) Patent Application Publication
(10) Pub. No.: US 2008/0154328 A1
(43) Pub. Date: Jun. 26, 2008

(54) UNIVERSAL CONNECTOR FOR IMPLANTABLE MEDICAL DEVICE

(75) Inventors: Todd THOMPSON, San Jose, CA (US); Benedict J. COSTELLO, Berkeley, CA (US); George M. SAVAGE, Portola Valley, CA (US)

Correspondence Address: BOZICEVIC, FIELD & FRANCIS LLP;(PROTEUS BIOMEDICAL, INC), 1900 UNIVERSITY AVENUE, SUITE 200, EAST PALO ALTO, CA, 94303, US

(73) Assignee: PROTEUS BIOMEDICAL, INC., Redwood City, CA (US)

(21) Appl. No.: 11/957,137

(22) Filed: Dec. 14, 2007

Related U.S. Application Data
(60) Provisional application No. 60/870,213, filed on Dec. 15, 2006.

(51) Int. Cl. A61N 1/375

(52) U.S. Cl. 607/37

(57) ABSTRACT

Implantable connector arrangements are provided for allowing a plurality of electrode leads to be connected to an implantable device through a single port in the device. Also provided are leads that include the same, implantable pulse generators that include the leads, as well as systems and kits having components thereof, and methods of making and using the subject devices.

### CROSS-REFERENCE TO RELATED APPLICATIONS

This application claims the benefit under 35 U.S.C. § 119(e) of prior U.S. provisional application Ser. No. 60/870,213 filed Dec. 15, 2006, disclosure of which applications is herein incorporated by reference.

### INTRODUCTION

Pacemakers and other implantable medical devices find wide-spread use in today's health care system. A typical pacemaker includes stimulating electrodes that are placed in contact with heart muscle, detection electrodes placed to detect movement of the heart muscle, and control circuitry for operating the stimulating electrodes based on signals received from the detection electrodes. Thus, the pacemaker can detect abnormal (e.g., irregular) movement and deliver electrical pulses to the heart to restore normal movement.

Pacing and/or sensing leads implanted in vessels in the body are, for many applications, flexible cylindrical devices. They are cylindrical due to three main reasons: most anatomical conduits are cylindrical, medical sealing and access devices seal on cylindrical shapes and cylindrical leads have uniform bending moments of inertia around the long axis of the device. These leads often have more than one electrode. One reason for placing multiple electrodes on a pacing and/or sensing lead is to help ensure that at least one electrode is contacting a desired stimulation or sensing point.

Due to the tortuous nature of the vessels in the body, following implantation the rotational orientation of one electrode can not be predetermined in many currently employed devices. As such, many currently employed lead devices employ cylindrical electrode designs that are conductive to tissue around the entirety of the diameter of the lead. This insures that some portion of the cylindrical electrode contacts excitable tissue or tissue of interest when they are implanted. Despite the multiple devices in which cylindrical continuous ring electrodes are employed, there are disadvantages to such structures, including but not limited to: undesirable excitation of non-target tissue, e.g., which can cause unwanted side effects, increased power use, etc.

An innovative way to address this problem is to employ segmented electrode structure, in which the circular band electrode is replaced by an electrode structure made up of two or more individually activatible and electrically isolated electrode structures that are configured in a discontinuous band. Such segmented electrode structures are disclosed in published PCT application Publication Nos. WO 2006/069322 and WO2006/029090; the disclosures of which are herein incorporated by reference.

Even with the multiple electrode structures on a pacing and/or sensing lead and with the multiple segments on each electrode structure described above, it may be discovered after a lead is implanted that an optimal stimulation and/or sensing location may not lie within the originally targeted vein in which the lead currently resides. Until now, this problem has had less than ideal solutions. One solution has been to attempt to continue use of an existing lead with its sub-optimal electrode placement. Another solution has been to change the location or orientation of the lead, which typically requires another surgical procedure along with its attendant risks and costs.

### SUMMARY

The present invention provides the ability to place additional electrode leads into a subject during surgery without increasing the size of the pacemaker or other implantable device. The additional electrodes help ensure that there is sufficient contact with optimal stimulation and/or sensing locations. As such, the present invention provides implantable devices that include satellite electrodes which can be implanted and maintain performance for long periods of time.

Embodiments of the invention include a universal connector, permitting multiple electrode leads to be connected to an implantable device through a single connection. Also provided are electrode leads that include the same, implantable pulse generators that include the leads, as well as systems and kits having components thereof, and methods of making and using the subject devices.

### BRIEF DESCRIPTION OF THE FIGURES

FIG. 1 provides a foreshortened plan view of an electrode lead used in conjunction with some embodiments of the invention;

FIG. 2 provides foreshortened plan view of connector arrangement including two of the electrode leads shown in FIG. 1;

FIG. 3 provides a diagrammatic isometric view of one exemplary embodiment of the invention;

FIG. 4 provides a diagrammatic isometric view of another exemplary embodiment of the invention;

FIG. 5 provides a plan view of another exemplary embodiment of the invention;

FIG. 6 provides a plan view of another exemplary embodiment of the invention;

FIG. 7 provides a plan view of another exemplary embodiment of the invention;

FIG. 8 provides a plan view of another exemplary embodiment of the invention;

FIG. 9 illustrates an exemplary external view of a number of pacing satellites on an electrode lead; and

FIG. 10 provides a depiction of a cardiac resynchronization therapy system that includes a connector 30 according to an embodiment of the invention.

### DETAILED DESCRIPTION

As summarized above, the present invention provides the ability to place additional electrode leads into a subject during surgery without increasing the size of the pacemaker or other implantable device. The additional electrodes help ensure that there is sufficient contact with optimal stimulation and/or sensing locations. As such, the present invention provides implantable devices that include satellite electrodes which can be implanted and maintain performance for long periods of time. Embodiments of the invention include a universal connector, permitting multiple electrode leads to be connected to an implantable device through a single connection. Also provided are electrode leads that include the same, implantable pulse generators that include the leads, as well as systems and kits having components thereof, and methods of making and using the subject devices.

In further describing aspects of the invention in greater detail, embodiments of the connector structures are reviewed first in greater detail. Next, a review of electrode leads and configurations that include the connector structures, as well as medical carriers and medical devices that include the same is provided. In addition, a further description of kits and systems of the invention, and methods of using various aspects of the invention, is provided.

### Connector Structures

As summarized above, aspects of the invention provide a connector. What is meant by connector is an implantable structure that physically and electrically interconnects two implantable devices or objects. For example, a pacing lead may be connected to a pacemaker with one or more connectors, or two or more pacing leads may be connected to each other with one or more connectors. A connector may be a separate item, or it may be integrally formed on another item, such as a pacing lead. What is meant by universal connector is a connector of a type used to interchangeably connect a variety of different items together, as apposed to a propriety connector used to connect only a specific item to another specific item. For example, a universal connector may be used to alternately connect one of a variety of pacing leads with one of a variety of pace makers. A universal connector may employ an industry standard connector type, such as those complying with a standard created, for example, by the International Standards Organization (ISO). In some embodiments of the present invention, a universal connector uses connector ports having an IS-1 designation, indicating they have been manufactured in compliance with ISO standard 5841.3:1992. In other embodiments, connector ports may have a DF-1 designation, indicating they have been manufactured in compliance with ISO standard 11318:1993. While the exemplary embodiments taught herein may describe universal connectors complying with the above standards, it is to be understood that certain aspects of the present invention are not limited to connectors employing such standards. The exemplary embodiments also depict the use of male plugs interconnecting with female sockets. However, other arrangements may also be used, according to aspects of the present invention. For example, planar connector elements that slidably interconnect or other connector structures may be used.

In certain embodiments, the connectors are configured to connect leads having “addressable” electrode structures. Addressable electrode structures include structures having one or more electrode elements coupled to control circuitry, e.g., present on an integrated circuit (IC). With addressable electrode structures, not all of the electrode segments of a particular electrode structure, and/or not all of the electrode structures on a lead need to be used at once. Rather, each electrode or electrode structure may be individually addressed so that it may be used independently from the others. According to aspects of the present invention, multiple leads may now be connected together and/or concurrently connected to a device such as a pacemaker, allowing individual electrodes and/or electrode segments on multiple leads to be individually addressed and operated. Examples of addressable electrode structures that include an IC are disclosed in application Ser. Nos.: 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various addressable electrode structures of these applications being herein incorporated by reference.

An implantable electrical connector assemblies of the invention include at least three input/output structures. The term “input/output structure” refers to a structure that is either an input structure or an output structure, as further defined below. In connector assemblies of the invention, each of the at least three input/output structures are electrically connected to each other. Furthermore, at least one of the input/output structures is an input structure and at least one of the input/output structures is an output structure.

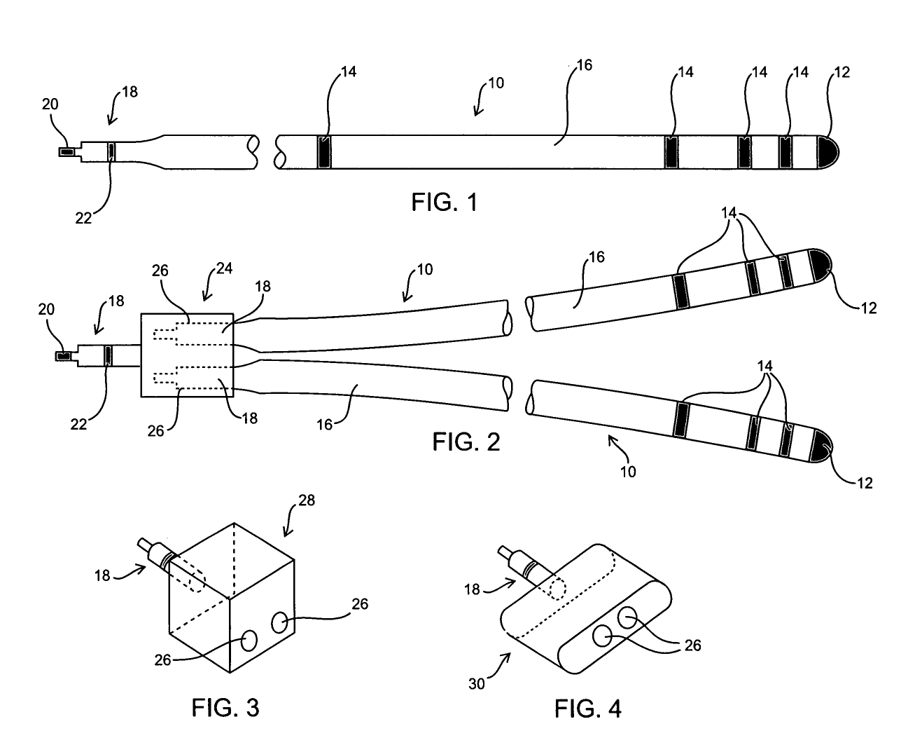

Referring to FIG. 1, an exemplary electrode lead 10 is shown. In this embodiment, lead 10 includes a tip electrode 12 and a plurality of ring electrodes 14 mounted on a flexible, elongate body 16. Each electrode 12 and 14 may be a single electrode or segmented into sub-electrodes. Electrodes 12 and 14 may be used for electrophysiology sensing, cardiac pacing and/or other purposes, as is well known in the art.

An input structure, such as a male plug 18 may be located at a proximal end for interconnecting lead 10 with an output structure of another device, such as a pacemaker. In this embodiment, input plug 18 includes a tip contact 20 and ring contact 22. While FIG. 1 depicts four distinct electrical contacts, other embodiments may employ one, two, three or more than four contacts. Other contact configurations may also be employed, such as separate pins for each contact. Contacts 20 and 22 may be electrically connected to electrodes 12 and 14, such as by wires running within lead body 16. In some embodiments, contacts 20 and 22 may be electrically connected directly with electrodes 12 and 14, such as in a one-to-one correspondence. In other embodiments, some or all of contacts 20 and 22 may be connected to a signal and/or power bus within lead body 16. Electrical circuits may be provided along the bus(es) to connect each electrode to the bus(es). With such an arrangement, each electrode or electrode segment may be addressed and activated separately. Such addressable electrode arrangements may reduce the number of contacts and/or connectors required to operate lead 10. These types of arrangements are described in detail in the references previously discussed above.

Referring to FIG. 2, a connector 24 according to an embodiment of the invention is shown. Connector 24 allows two leads 10 to be connected to a single port, such as the electrical output port of a pacemaker. The input plug 18 of each lead 10 is received within its own output port 26 of connector 24. Each of the two output ports 26 of connector 24 replicate the output port of the pacemaker. As such, each output structure is configured to mate with a structure similar to the input structure. As shown, each of the at least two output structures are offset from another output structure in at least two dimensions. In certain embodiments, each contact of each output port 26 is electrically connected in parallel to the corresponding contact on input plug 18 of connector 24, such that each output structure includes at least one contact that is electrically connected to a corresponding contact on the input structure. Plug 18 of connector 24 may be plugged directly into the port of a pacemaker or other device, thereby acting as a splitter and allowing two leads 10 to be connected in parallel to the device simultaneously. Alternatively, a cable having one male and one female end may be used to join connector 24 to the port of a device. In certain embodiments, leads 10 each have addressable electrodes, allowing each electrode or electrode segment on each lead 10 to be used independently of the others. Connector 24 as shown has smooth contours and rounded corners (not shown) so that damage is not caused to body tissue when connector 24 is implanted.

With the above arrangements, a device such as a pacemaker need not have multiple ports to use multiple leads 10. According to aspects of the invention, this allows multiple electrode leads to be used without redesigning the device and/or making it larger. According to other aspects, these arrangements also permit a surgeon to spread the extra volume associated with multiple ports to other locations within the body, rather than locating the entire mass in one location (i.e. all at the location of an implanted device.)

Referring to FIG. 3, another connector embodiment is diagrammatically shown. Connector 28 may be constructed and used in a similar fashion to connector 24 shown in FIG. 2, and has two ports 26. Accordingly, two electrode leads 10 may be coupled through plug 18 of connector 28 to a single port in a device or cable. In other embodiments, fewer or more than two ports 26 may be employed by a connector. Dummy plugs (not shown) may be employed to fill any unused ports 26 to electrically and physically isolate such ports after implantation. Again, rounded corners and edges (not shown) may be used to make connector 28 more amenable to residing in a living body.

Referring to FIG. 4, another exemplary connector embodiment is shown. Connector 30 has two ports 26 like the previous embodiment, but in this example ports 26 are arranged in a flat array. In other embodiments, ports 26 may be arranged in a triangle, circle, arc, trapezoid, parallelogram or other arrangements. Ports 26 need not all be parallel as shown, but may be oriented in different directions and emanate from different surfaces of the connector. Ports 26 may also be staggered in axial and/or radial directions. Reasons for such arrangements may include making the connector more compact and/or conforming the connector to the shape of a particular void within a subject's body.

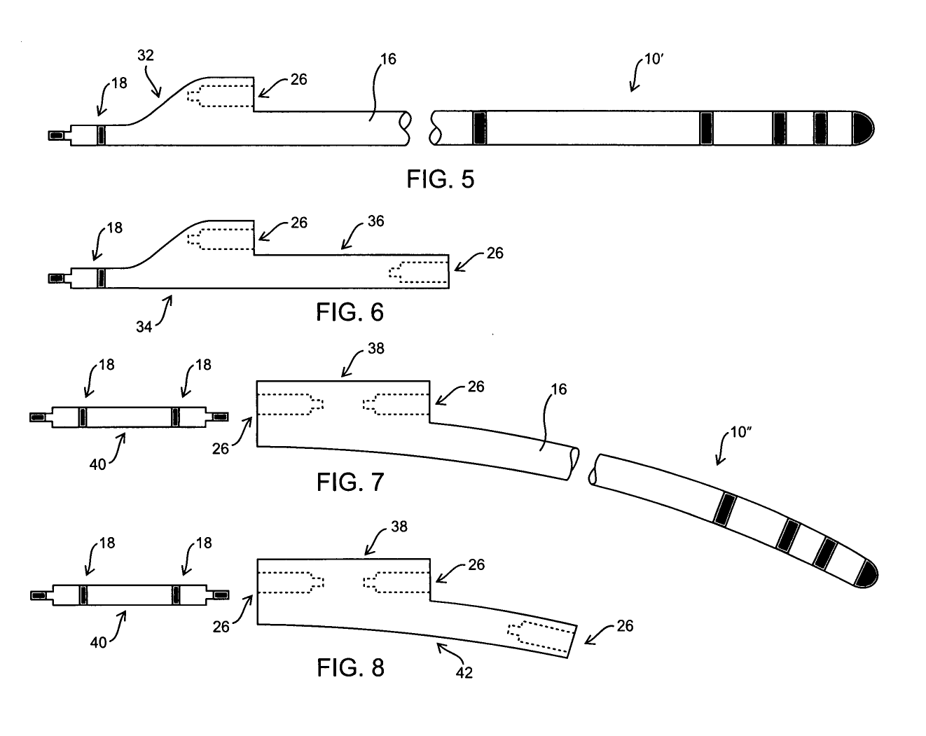

Referring to FIG. 5, an electrode lead 10′ is shown having a connector integrally formed on its proximal end. Housing 32 may be formed on or coupled to lead 10′ to accommodate port 26. The embodiment shown in FIG. 5 provides a compact arrangement for utilizing two or more electrode leads. It is similar to the arrangement shown in FIG. 2, but uses one less port 26 and one less plug 18 (i.e. half as many), thereby reducing the size and cost of the lead arrangement, and increasing its reliability. As shown in FIG. 5, input plug 18 and output port 26 face the same direction, allowing a lead 10′ received in port 26 to extend in the same direction as lead 10′. Port 26 may alternatively be oriented in the opposite direction such that it faces plug 18, orthogonally so that it is perpendicular rather than parallel to body 16 of lead 10′, or any other suitable orientation. Housing 32 and port 26 need not be placed adjacent to plug 18 at the distal end of lead 10′, but may alternately be placed at another location along body 16. In other embodiments, two or more ports 26 located in one or more housings 32 may be located at various positions along body 16 of lead 10.

The arrangement shown in FIG. 5 and described above allows an electrode lead 10 or 10′ to be received in port 26 shown. When another lead of type 10′ is used (i.e. another lead having an additional port 26), three or more leads may then be “daisy chained” together and connected to a device through a single plug 18. Unless the last lead in the chain is of type 10 (i.e. a lead not having a port 26), a dummy plug or suitable cover may be used to close the open end of port 26 in the last lead 10′.

Referring to FIG. 6, an alternative embodiment is shown. Connector 34 is similar in construction and operation to the arrangement shown in FIG. 5 except that electrode lead 10′ is replaced with a portion 36 having a second port 26. Connector portion 36 may be flexible and constructed similarly to leads 10 and 10′. Alternatively, portion 36 may be semi-rigid or rigid, and/or may be integrally formed with the rest of connector 34. Portion 36 may have a length of about 3 cm to about 15 cm, such as from about 4 to about 12 cm, and including from about 5 to about 20 cm. In this embodiment, input plug 18 and the two output ports 26 are generally parallel with each other and facing the same direction. One of the ports 26 is generally aligned with plug 18, and the other port 26 is radially (i.e. laterally) offset from plug 18 and the aligned port 26. The radially offset port 26 may also be offset in an axial (i.e. longitudinal) direction from the other port 26. The above arrangement spreads out the bulk, and in some embodiments the stiffness, associated with the three connectors 18, 26 and 26, which may aid in implanting connector 24 and related devices. Since connector 34 of this embodiment has one port 26 aligned with plug 18 and the other port 26 radially offset by a small amount, connector 34 may be used to connect two leads 10 to a single output port of a device without occupying much more room inside a patient than the volume of the leads 10 themselves.

Referring to FIG. 7, an alternative embodiment is shown. In this embodiment, electrode lead 10″ has a housing 38 formed on or coupled to its proximal end. Housing 38 includes two ports 26, preferably facing in opposite directions. With this arrangement, lead 10″ may be connected “in line” with one or more other leads 10, 10′ or 10″ in a compact fashion. Connector 40, having a plug 18 at each end, may be used to connect lead 10″ to an implantable device, another connector, cable or lead.

Referring to FIG. 8, an alternative embodiment is shown. This embodiment is similar to the embodiment shown in FIG. 7 except that electrode lead 10″ is replaced with a portion 42 having a third port 26. Connector portion 42 may be flexible and constructed similarly to leads 10 and 10′. Alternatively, portion 42 may be semi-rigid or rigid, and/or may be integrally formed with housing 38. In certain embodiments, portion 42 has a length of about 3 cm to about 20 cm, such as from about 5 cm to about 15 cm and including from about 7 to about 12 cm.

The above exemplary embodiments may be used as described or used in various combinations and/or variations. For example, plugs 18 may be substituted for ports 26 in some situations, and vice versa. Separate items may be combined, and/or single items may be made into separate items. Rigid items may be made flexible, and vice versa. For example, the embodiment of FIG. 2 may be modified such that connector 24 is essentially a Y-cable having a port 26 at the end of each of the top legs and a plug 18 at the end of the bottom leg.

As summarized above, the invention provides implantable medical devices that include the connector structures as described above. By implantable medical device is meant a device that is configured to be positioned on or in a living body, where in certain embodiments the implantable medical device is configured to be implanted in a living body. Embodiments of the implantable devices are configured to maintain functionality when present in a physiological environment, including a high salt, high humidity environment found inside of a body, for 2 or more days, such as about 1 week or longer, about 4 weeks or longer, about 6 months or longer, about 1 year or longer, e.g., about 5 years or longer. In certain embodiments, the implantable devices are configured to maintain functionality when implanted at a physiological site for a period ranging from about 1 to about 80 years or longer, such as from about 5 to about 70 years or longer, and including for a period ranging from about 10 to about 50 years or longer. The dimensions of the implantable medical devices of the invention may vary. However, because the implantable medical devices are implantable, the dimensions of certain embodiments of the devices are not so big such that the device cannot be positioned in an adult human.

### Vascular Leads

Embodiments of the invention also include medical carriers that include one or more electrode and connector structures, e.g., as described above. Carriers of interest include, but are not limited to, vascular lead structures, where such structures are generally dimensioned to be implantable and are fabricated from a physiologically compatible material. With respect to vascular leads, a variety of different vascular lead configurations may be employed, where the vascular lead in certain embodiments is an elongated tubular, e.g., cylindrical, structure having a proximal and distal end. The proximal end may include a connector element, e.g., an IS-1 or DF-1 connector, for connecting to a control unit, e.g., present in a “can” or analogous device. The lead may include one or more lumens, e.g., for use with a guidewire, for housing one or more conductive elements, e.g., wires, etc. The distal end may include a variety of different features as desired, e.g., a securing means, a particular configuration, e.g., S-bend, etc.

In certain embodiments of the subject systems, one or more sets of electrode and connector structures as described above are electrically coupled to at least one elongated conductive member, e.g., an elongated conductive member present in a lead, such as a cardiovascular lead. In certain embodiments, the elongated conductive member is part of a multiplex lead. Multiplex lead structures may include 2 or more satellites, such as 3 or more, 4 or more, 5 or more, 10 or more, 15 or more, 20 or more, etc. as desired, where in certain embodiments multiplex leads have a fewer number of conductive members than satellites. In certain embodiments, the multiplex leads include 3 or less wires, such as only 2 wires or only 1 wire. Multiplex lead structures of interest include those described in application Ser. Nos.: 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various multiplex lead structures of these applications being herein incorporated by reference. In some embodiments of the invention, the devices and systems may include onboard logic circuitry or a processor, e.g., present in a central control unit, such as a pacemaker can. In these embodiments, the central control unit may be electrically coupled to the lead by one or more of the connector arrangements described above.

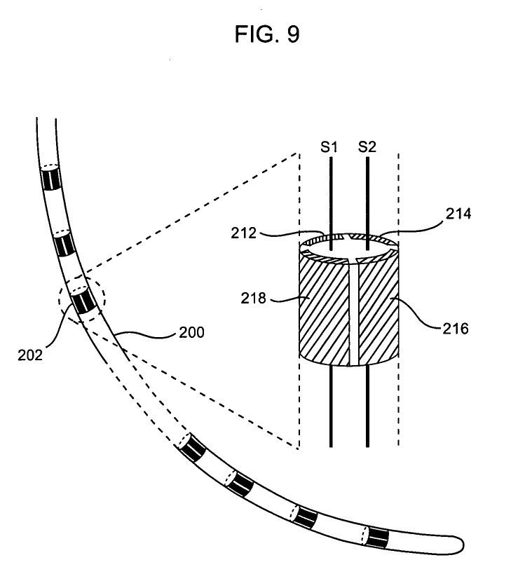

FIG. 9 illustrates an external view of a number of exemplary pacing satellites, in accordance with a multiplex lead embodiment of the present invention. According to one embodiment, a pacing lead 200 (e.g., right ventricular lead 109 or left ventricular lead 107 of FIG. 12) accommodates two bus wires S1 and S2, which are coupled to a number (e.g., eight) of satellites, such as satellite 202. FIG. 9 also shows satellite 202 with an enlarged view. Satellite 202 includes electrodes 212, 214, 216, and 218, located in the four quadrants of the cylindrical outer walls of satellite 202 and supported by a support structure of the invention. Each satellite also contains a control chip inside the structure which communicates with a pacing and signal-detection system to receive configuration signals that determine which of the four electrodes are to be coupled to bus wires S1 or S2. Bus wires S1 and S2 in turn may be coupled to an implantable device such as a pacemaker through one or more of the connector arrangements described above.

The configuration signals, the subsequent pacing pulse signals, and the analog signals collected by the electrodes can all be communicated through bus wires S1 and S2, in either direction. Although shown in a symmetrical arrangement, electrodes 212, 214, 216 and 218 may be offset along lead 200 to minimize capacitive coupling among these electrodes. The quadrant arrangement of electrodes allows administering pacing current via electrodes oriented at a preferred direction, for example, away from nerves, or facing an electrode configured to sink the pacing current. Such precise pacing allows low-power pacing and minimal tissue damage caused by the pacing signal.

The leads may further include a variety of different effector elements, which elements may employ the satellites or structures distinct from the satellites. The effectors may be intended for collecting data, such as but not limited to pressure data, volume data, dimension data, temperature data, oxygen or carbon dioxide concentration data, hematocrit data, electrical conductivity data, electrical potential data, pH data, chemical data, blood flow rate data, thermal conductivity data, optical property data, cross-sectional area data, viscosity data, radiation data and the like. As such, the effectors may be sensors, e.g., temperature sensors, accelerometers, ultrasound transmitters or receivers, voltage sensors, potential sensors, current sensors, etc. Alternatively, the effectors may be intended for actuation or intervention, such as providing an electrical current or voltage, setting an electrical potential, heating a substance or area, inducing a pressure change, releasing or capturing a material or substance, emitting light, emitting sonic or ultrasound energy, emitting radiation and the like.

Effectors of interest include, but are not limited to, those effectors described in the following applications by at least some of the inventors of the present application: U.S. patent application Ser. No. 10/734,490 published as 20040193021 titled: “Method And System For Monitoring And Treating Hemodynamic Parameters”; U.S. patent application Ser. No. 11/219,305 published as 20060058588 titled: “Methods And Apparatus For Tissue Activation And Monitoring”; International Application No. PCT/US2005/046815 titled: “Implantable Addressable Segmented Electrodes”; U.S. patent application Ser. No. 11/324,196 titled “Implantable Accelerometer-Based Cardiac Wall Position Detector”; U.S. patent application Ser. No. 10/764,429, entitled “Method and Apparatus for Enhancing Cardiac Pacing,” U.S. patent application Ser. No. 10/764,127, entitled “Methods and Systems for Measuring Cardiac Parameters,” U.S. patent application Ser. No. 10/764,125, entitled “Method and System for Remote Hemodynamic Monitoring”; International Application No. PCT/US2005/046815 titled: “Implantable Hermetically Sealed Structures”; U.S. application Ser. No. 11/368,259 titled: “Fiberoptic Tissue Motion Sensor”; International Application No. PCT/US2004/041430 titled: “Implantable Pressure Sensors”; U.S. patent application Ser. No. 11/249,152 entitled “Implantable Doppler Tomography System,” and claiming priority to: U.S. Provisional Patent Application No. 60/617,618; International Application Serial No. PCT/USUS05/39535 titled “Cardiac Motion Characterization by Strain Gauge”. These applications are incorporated in their entirety by reference herein.

### Implantable Pulse Generators

Embodiments of the invention further include implantable pulse generators. Implantable pulse generators may include: a housing which includes a power source and an electrical stimulus control element; one or more vascular leads as described above, e.g., 2 or more vascular leads, where each lead is coupled to the control element in the housing via a suitable connector or connectors as described above. In certain embodiments, the implantable pulse generators are ones that are employed for cardiovascular applications, e.g., pacing applications, cardiac resynchronization therapy applications, etc. As such, in certain embodiments the control element is configured to operate the pulse generator in a manner so that it operates as a pacemaker, e.g., by having an appropriate control algorithm recorded onto a computer readable medium of a processor of the control element. In certain embodiments the control element is configured to operate the pulse generator in a manner so that it operates as a cardiac resynchronization therapy device, e.g., by having an appropriate control algorithm recorded onto a computer readable medium of a processor of the control element.

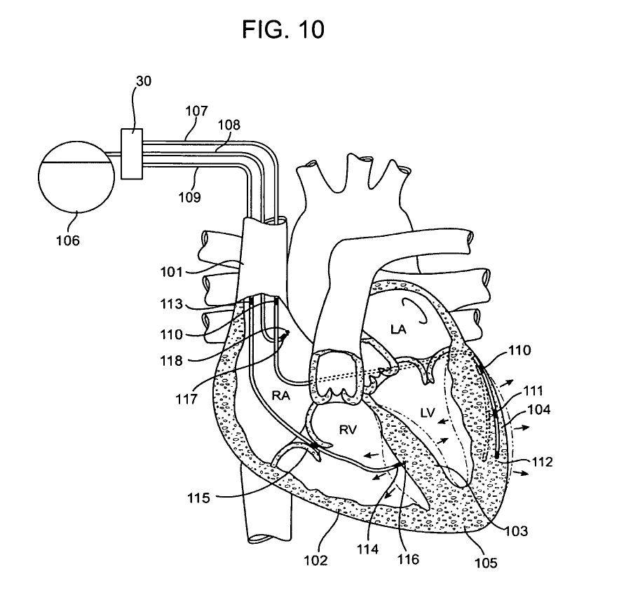

An implantable pulse generator according to an embodiment of the invention is depicted in FIG. 10, which provides a cross-sectional view of the heart with of an embodiment of a cardiac resynchronization therapy (CRT) system. The system includes a pacemaker can 106 that includes a control element (e.g., processor) and a power source, a right ventricle electrode lead 109, a right atrium electrode lead 108, and a left ventricle cardiac vein lead 107. Also shown are the right ventricle lateral wall 102, interventricular septal wall 103, apex of the heart 105, and a cardiac vein on the left ventricle lateral wall 104.

The left ventricle electrode lead 107 is comprised of a lead body and one or more satellite electrode assemblies 110,111, and 112. Each of the electrodes assemblies is a satellite as described above and includes a hermetically sealed integrated circuit electrically coupled to four distinct electrode element arranged in a quadrant configuration. Having multiple distal electrode assemblies allows a choice of optimal electrode location for CRT. In a representative embodiment, electrode lead 107 is constructed with the standard materials for a cardiac lead such as silicone or polyurethane for the lead body, and MP35N for the coiled or stranded conductors connected to Pt—Ir (90% platinum, 10% iridium) electrode assemblies 110,111 and 112. Alternatively, these device components can be connected by a multiplex system (e.g., as described in published United States Patent Application publication nos.: 20040254483 titled “Methods and systems for measuring cardiac parameters”; 20040220637 titled “Method and apparatus for enhancing cardiac pacing”; 20040215049 titled “Method and system for remote hemodynamic monitoring”; and 20040193021 titled “Method and system for monitoring and treating hemodynamic parameters; the disclosures of which are herein incorporated by reference), to the proximal end of electrode lead 107. The proximal end of electrode lead 107 connects to a pacemaker 106, e.g., via an IS-1 connector.

The electrode lead 107 is placed in the heart using standard cardiac lead placement devices which include introducers, guide catheters, guidewires, and/or stylets. Briefly, an introducer is placed into the clavicle vein. A guide catheter is placed through the introducer and used to locate the coronary sinus in the right atrium. A guidewire is then used to locate a left ventricle cardiac vein. The electrode lead 107 is slid over the guidewire into the left ventricle cardiac vein 104 and tested until an optimal location for CRT is found. Once implanted a multi-electrode lead 107 still allows for continuous readjustments of the optimal electrode location.

The electrode lead 109 is placed in the right ventricle of the heart with an active fixation helix at the end 116 which is embedded into the cardiac septum. In this view, the electrode lead 109 is provided with one or multiple electrodes 113,114,115.

Electrode lead 109 is placed in the heart in a procedure similar to the typical placement procedures for cardiac right ventricle leads. Electrode lead 109 is placed in the heart using the standard cardiac lead devices which include introducers, guide catheters, guidewires, and/or stylets. Electrode lead 109 is inserted into the clavicle vein, through the superior vena cava, through the right atrium and down into the right ventricle. Electrode lead 109 is positioned under fluoroscopy into the location the clinician has determined is clinically optimal and logistically practical for fixating the electrode lead 109. Under fluoroscopy, the active fixation helix 116 is advanced and screwed into the cardiac tissue to secure electrode lead 109 onto the septum. The electrode lead 108 is placed in the right atrium using an active fixation helix 118. The distal tip electrode 118 is used to both provide pacing and motion sensing of the right atrium.

Summarizing aspects of the above description, in using the implantable pulse generators of the invention, such methods include implanting an implantable pulse generator e.g., as described above, into a subject; and the implanted pulse generator, e.g., to pace the heart of the subject, to perform cardiac resynchronization therapy in the subject, etc. The description of the present invention is provided herein in certain instances with reference to a subject or patient. As used herein, the terms “subject” and “patient” refer to a living entity such as an animal. In certain embodiments, the animals are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the subjects, e.g., patients, are humans.

During operation, use of the implantable pulse generator may include activating at least one of the electrodes of the pulse generator to deliver electrical energy to the subject, where the activation may be selective, such as where the method includes first determining which of the electrodes of the pulse generator to activate and then activating the electrode. Methods of using an IPG, e.g., for pacing and CRT, are disclosed in Application Serial Nos.: PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various methods of operation of these applications being herein incorporated by reference and applicable for use of the present devices.

### Systems

Also provided are systems that include one more devices as described above, an implantable pulse generator. The systems of the invention may be viewed as systems for communicating information within the body of subject, e.g., human, where the systems include both a first implantable medical device, such as an IPG device described above, that includes a transceiver configured to transmit and/or receive a signal; and a second device comprising a transceiver configured to transmit and/or receive a signal. The second device may be a device that is inside the body, on a surface of the body or separate from the body during use.

Also provided are methods of using the systems of the invention. The methods of the invention generally include: providing a system of the invention, e.g., as described above, that includes first and second medical devices, one of which may be implantable; and transmitting a signal between the first and second devices. In certain embodiments, the transmitting step includes sending a signal from the first to said second device. In certain embodiments, the transmitting step includes sending a signal from the second device to said first device. The signal may transmitted in any convenient frequency, wherein certain embodiments the frequency ranges from about 400 to about 405 MHz. The nature of the signal may vary greatly, and may include one or more data obtained from the patient, data obtained from the implanted device on device function, control information for the implanted device, power, etc.

Use of the systems may include visualization of data obtained with the devices. Some of the present inventors have developed a variety of display and software tools to coordinate multiple sources of sensor information which will be gathered by use of the inventive systems. Examples of these can be seen in international PCT application serial no. PCT/US2006/012246; the disclosure of which application, as well as the priority applications thereof are incorporated in their entirety by reference herein.

### Methods of Making

The subject structures and devices described herein may be fabricated using any convenient protocol. Aspects of the invention include methods of making a vascular electrode lead and/or connector assembly, as described above.

### Kits

Also provided are kits that include the subject electrode lead and/or connector structures, as part of one or more components of an implantable device or system, such as an implantable pulse generator, e.g., as reviewed above. In certain embodiments, the kits further include at least a control unit, e.g., in the form of a pacemaker can. In certain of these embodiments, at least some of the electrodes in the system are coupled to the control unit with one or more connector arrangements as described above.

In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

It is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. An implantable electrical connector assembly comprising at least three input/output structures, wherein each of said at least three input/output structures are electrically connected to each other, at least one of said input/output structures is an input structure and at least one of said input/output structures is an output structure.

2. The implantable electrical connector assembly according to claim 1, wherein said connector comprises:
an input structure; and
at least two output structures, wherein each output structure is configured to mate with a structure similar to the input structure, wherein each output structure includes at least one contact that is electrically connected to a corresponding contact on the input structure, and wherein each of the at least two output structures are offset from another output structure in at least two dimensions.

3. The connector assembly of claim 2, wherein there are exactly two output structures, the two output structures being parallel to each other.

4. The connector assembly of claim 3, wherein one of the output structures is in line with the input structure.

5. The connector assembly of claim 2, further comprising a body that houses each of the input and output structures, the body having a substantially rigid portion and a substantially flexible portion.

6. The connector assembly of claim 2, wherein the input structure is a pin-shaped plug and the output structures are socket-shaped ports.

7. An implantable electrical connector according to claim 2, wherein the first output structure has a longitudinal axis aligned with a longitudinal axis of the input structure, and wherein the second output structure is radially offset from the first output structure.

8. An implantable lead structure comprising:
an elongated flexible body including a proximal end, a distal end and a longitudinal axis;
at least one electrode assembly located on the lead body;
an input structure located at the proximal end of the lead body; and
at least one output structure located on the lead body radially offset from the longitudinal axis, wherein the output structure is configured to mate with a structure similar to the input structure, wherein the output structure includes at least one contact that is electrically connected to a corresponding contact on the input structure.

9. The lead structure according to claim 8, wherein said structure is a vascular lead.

10. The lead structure according to claim 9, wherein said vascular lead comprises 2 or more electrodes.

11. The lead structure according to claim 10, wherein said vascular lead is a multiplex lead having 3 or less wires.

12. The lead structure according to claim 11, wherein said vascular lead includes only 2 wires.

13. The lead structure according to claim 12, wherein said vascular lead includes only 1 wire.

14. The lead structure according to claim 9, wherein the input structure includes an IS-1 connector.

15. The lead structure of claim 8, wherein the input structure is a pin-shaped plug and the output structure is a socket-shaped port.

16. The lead structure of claim 8, wherein the input structure and the output structure are socket-shaped ports.

17. The lead structure of claim 8, further comprising two or more output structures.

18. A lead assembly comprising:
(a) implantable electrical connector assembly comprising:
(i) an input structure; and
(ii) at least two output structures, wherein each output structure is configured to mate with a structure similar to the input structure, wherein each output structure includes at least one contact that is electrically connected to a corresponding contact on the input structure, and wherein each of the at least two output structures are offset from another output structure in at least two dimensions; and
(b) a vascular lead connected to an output structure of said connector assembly.

19. An implantable pulse generator comprising:
(a) a housing comprising a power source and an electrical stimulus control element; and
(b) a lead according to claim 8 or a lead assembly according to claim 18.

20. The implantable pulse generator according to claim 19, wherein said control element is configured to operate said implantable pulse generator as a pacemaker.

21. The implantable pulse generator according to claim 19, wherein said control element is configured to operate said implantable pulse generator in a manner sufficient to achieve cardiac resynchronization.

22. A system comprising:
(a) a first implantable pulse generator according to claim 19; and
(b) a second device configured to communicate with said implantable pulse generator.

23. The system according to claim 22, wherein said second device is an implantable medical device.

24. A method comprising:
implanting an implantable pulse generator according to claim 19 into a subject; and
using said implanted pulse generator.

25. The method according to claim 24, wherein said using comprises activating an electrode of said pulse generator to deliver electrical energy to said subject.

26. The method according to claim 24, wherein said method further comprises determining which electrode of said pulse generator to activate.

27. A method comprising:
implanting a housing comprising a control unit and a power element into a patient;
electrically coupling at least one multiplex lead to said control unit using a connector comprising a single input structure and at least two output structures; and
operating said control unit to deliver an electrical pulse to said patient via said multiplex lead.

28. The method according to claim 27, wherein said method comprises electrically coupling at least two multiplex leads to said control unit using said connector.

29. The method according to claim 27, wherein said multiplex lead comprises a segmented electrode.

30. A kit comprising:
(a) a connector comprising a single input structure and at least two output structures; and
(b) a multiplex vascular lead.
